(19) United States
(12) Patent Application Publication
(10) Pub. No.: US 2014/0148816 A1
(43) Pub. Date: May 29, 2014

(54) SURGERY PORT PLACEMENT SYSTEM AND RELATED METHODS

(71) Applicants: Michael W. McDONALD, Celebration, FL (US); Mark E. JOHNSON, Maitland, FL (US); Una Joyce COULTER, Celebration, FL (US)

(72) Inventors: Michael W. McDONALD, Celebration, FL (US); Mark E. JOHNSON, Maitland, FL (US); Una Joyce COULTER, Celebration, FL (US)

(21) Appl. No.: 14/090,563

(22) Filed: Nov. 26, 2013

Related U.S. Application Data
(60) Provisional application No. 61/729,673, filed on Nov. 26, 2012.

(51) Int. Cl. A61B 19/00

(52) U.S. Cl. CPC A61B 19/50; A61B 19/2203
USPC 606/130

(57) ABSTRACT

A surgical port placement system is to be adjacent a surgical table. The surgical port placement system may include a surgical port placement device for storing parameter sets respectively associated with past surgical procedures, generating a surgical port placement model based upon the parameter sets, receiving a given parameter set for a given surgical procedure for a given patient, the given parameter set having physical characteristics of the given patient, and generating a surgical port placement position for the given patient for the given surgical procedure based upon the given parameter set and the surgical port placement model.

### RELATED APPLICATIONS

This application is based upon prior filed copending application Ser. No. 61/729,673 filed Nov. 26, 2012, the entire subject matter of which is incorporated herein by reference in its entirety.

### FIELD OF THE INVENTION

The present invention relates to the field of medical treatment, and, more particularly, to surgical procedure and related devices.

### BACKGROUND OF THE INVENTION

Surgery in one form or another has been part of the medical treatment tool chest for over a thousand years. Of course, the earliest forms of surgery were not the most effective medical treatments. Indeed, before the scientific era and the advent of modern surgery, most surgery procedures likely did more harm than good for the patient.

As surgical techniques have improved, the negative effects on the patient, such as bleeding, pain, and infection, have been reduced. Nevertheless, certain surgical procedures, such as a laparotomy, by their very nature may present more risk to the patient since they require large incisions. An approach to overcome the risks of the laparotomy surgical procedure is laparoscopic surgery.

Laparoscopic surgery is performed on the same area as the laparotomy surgical procedure, i.e. the abdomen, but rather than requiring the surgeon to make a large incision in the patient, the surgeon makes a plurality of small incisions (surgical ports) in the general area where internal work is needed. Once the small incisions have been made, the surgeon may insert a laparoscope and one or more working instruments into the small incisions. The laparoscope may comprise an articulated rod comprising a plurality of lens, and a camera on the proximal end (i.e. the end outside of the patient). Another typical laparoscope may comprise a digital camera device on the distal end (i.e. the end inserted in the patient).

Quite sensibly, during laparoscopic surgery, the reach and operational effectiveness of the surgeon is based upon the efficient placement of the small incisions. For example, if the placement is poor, the laparoscope may not reach the needed areas or if multiple laparoscopes/instruments are used, they may crisscross. In typical laparoscopic surgeries, the placement of the small incisions or ports is “eyeballed” by the surgeon and based upon the surgeon's experience.

U.S. Patent Application No. 2012/0253515 to Coste-Maniere et al. discloses an approach to placing small incisions for laparoscopic surgery. This approach discloses a device for enhancing surgical planning of entry port placement and robot position for laparoscopic, robotic, and other minimally invasive surgery. Generally, imaging data is processed and used to create a model of a surgical site, which can then be used to select entry port sites for two or more surgical tools based on multiple criteria.

### SUMMARY OF THE INVENTION

In view of the foregoing background, it is therefore an object of the present invention to provide a system that efficiently suggests surgical port placement both for laparoscopic and robotic procedures.

This and other objects, features, and advantages in accordance with the present invention are provided by a surgical port placement system. The surgical port placement system may be adjacent a surgical table, and may include a surgical port placement device comprising a memory and processor cooperating therewith. The processor and memory may be for storing a plurality of parameter sets respectively associated with a plurality of past surgical procedures, generating a surgical port placement model based upon the plurality of parameter sets, receiving a given parameter set for a given surgical procedure for a given patient, the given parameter set comprising physical characteristics of the given patient, and generating at least one surgical port placement position for the given patient for the given surgical procedure based upon the given parameter set and the surgical port placement model. The surgical port placement system may include a direction tool coupled to the surgical port placement device and for indicating the at least one surgical port placement position on the given patient on the surgical table. Advantageously, the surgical port placement system provides surgical port placement positions that are efficient and enable easy access for the surgical procedure.

In some embodiments, the physical characteristics of the given patient may comprise positions for boney prominences on the given patient, a weight for the given patient, and a height for the given patient. The plurality of parameter sets respectively associated with the plurality of past surgical procedures may comprise the physical characteristics of a respective patient, and a plurality of performance metrics for a respective surgical port placement position for the respective patient.

More specifically, the generating of surgical port placement position for the given patient for the given surgical procedure may comprise generating a plurality of vectors, each vector extending from a physical point on the given patient to the at least one surgical port placement position. The generating of the surgical port placement model may comprise using a Naïve Bayes Learning machine to process the plurality of parameter sets respectively from the plurality of past surgical procedures.

Additionally, the surgical port placement device may comprise an input interface coupled to the processor for receiving the given parameter set for the given surgical procedure for the given patient. In some embodiments, the direction tool may comprise a light source for illuminating the at least one surgical port placement position on the given patient on the surgical table. In other embodiments, the direction tool may comprise a display monitor for indicating the at least one surgical port placement position on the given patient on the surgical table.

Another aspect is directed to a surgical port placement device. The surgical port placement device may include a memory and processor cooperating therewith and for storing a plurality of parameter sets respectively associated with a plurality of past surgical procedures, and generating a surgical port placement model based upon the plurality of parameter sets. The processor and memory may be for receiving a given parameter set for a given surgical procedure for a given patient, the given parameter set comprising physical characteristics of the given patient, and generating at least one surgical port placement position for the given patient for the given surgical procedure based upon the given parameter set and the surgical port placement model.

Another aspect is directed to a method for determining at least one surgical port placement position for a given patient for a given surgical procedure. The method may include storing a plurality of parameter sets respectively associated with a plurality of past surgical procedures, and generating a surgical port placement model based upon the plurality of parameter sets. The method may include receiving a given parameter set for the given surgical procedure for the given patient, the given parameter set comprising physical characteristics of the given patient, and generating at least one surgical port placement position for the given patient for the given surgical procedure based upon the given parameter set and the surgical port placement model.

### BRIEF DESCRIPTION OF THE DRAWINGS

FIG. 1 is a schematic diagram of a surgical port placement system, according to the present invention.

FIG. 2 is an image of measurements for surgical port placements, according to the present invention.

### DETAILED DESCRIPTION OF THE EMBODIMENTS

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

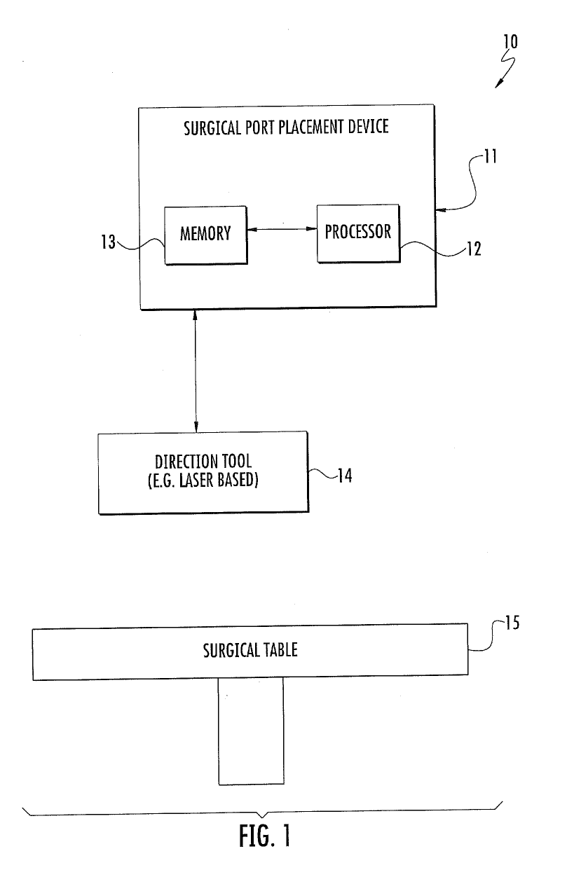

Referring now to FIG. 1, a surgical port placement system 10 according to the present invention is now described. The surgical port placement system 10 illustratively includes a surgical port placement device 11 comprising a memory 13, and a processor 12 cooperating therewith. For example, the surgical port placement device 11 may comprise a general purpose computing device modified by the teachings herein. The surgical port placement system 10 illustratively includes a direction tool 14 coupled to the surgical port placement device 11 and adjacent to a surgical table 15. In typical fashion, a patient is positioned on the surgical table 15 for a surgical procedure requiring surgical ports.

During the surgical procedure, the surgeon may utilize the surgical port placement system 10 to provide potential surgical port placement positions. The surgical port placement device 11 may comprise an input interface (not shown), such as a keyboard, a touch screen, or a voice recognition unit, and the surgeon may utilize the input interface to enter in patient characteristics. For example, the patient characteristics may comprise weight, body mass index (BMI), patient height, and operation region dimensions, such as patient chest width and height, as well as the particular organ/organs being treated and the location of a specific tumor to be operated on are to be included.

The surgical port placement device 11 stores within the memory 13 a statistical database regarding surgical port placement. The development of the statistical database is discussed in detail hereinbelow. In short, the statistical database comprises a plurality of data entries that describe past surgical procedures including surgical port placement positions, respective patient characteristics, and overall success of the surgical port placement. Using the patient characteristics entered by the surgeon, the surgical port placement device 11 may determine suggested surgical port placement positions for the intended surgical procedure using past performance statistics in the database. In some embodiments, even if the patient characteristics for the current patient are not common in the database (e.g. out of the typical range of data, i.e. an outlier, or a unique combination of characteristics), the processor 12 may utilize prediction algorithms to expand the applicable data range in the database and provide the suggested surgical port placement positions for the current patient.

In one embodiment (not shown), the surgical port placement device 11 comprises an output interface, such as display or audio speaker, and the suggested surgical port placement positions are displayed on the output interface for the surgeon. For example, the suggested surgical port placement positions may be based upon relative positions from bony prominences, i.e. providing a point (bony prominence), direction, and distance for the surgical port position.

In the illustrated embodiment, the suggested surgical port placement positions are actively noted on the patient by the direction tool 14. For example, the direction tool 14 may comprise a plurality of laser pointers, and a camera based position system (i.e. the camera system determines the position of the patient on the surgical table 15) and may indicate suggested surgical port placement positions with the laser pointers. In some embodiments, the processor 12 may cooperate with the camera system of the direction tool 14 to determine the patient characteristics automatically and without manual determination by the surgeon. Once the suggested surgical port placement positions have been provided to the surgeon, the surgeon may make the necessary incisions and insert one or more laparoscopes for surgery.

In some embodiments, the processor 12 may provide only a single suggested surgical port, i.e. robotic or laparoscopic single port sites. For example, the single surgical port would comprise an enlarged surgical port, such as being 3 cm in size, for inserting one or more laparoscopes for the surgery.

Another aspect is directed to a method of using the surgical port placement system 10 for a given surgical procedure. The method includes performing a plurality of surgical procedures using surgical ports, recording respective patient characteristics and surgical port placement positions, determining respective efficacy factors for each performed surgical procedure, and storing the respective patient characteristics, the surgical port placement positions, and the respective efficacy factors for the performed surgical procedures. The method also includes processing the stored data and performing statistical modeling to provide a formula. The formula may provide suggested surgical port placement positions based upon patient characteristics and the respective surgical procedure. The method also includes determining suggested surgical port placement positions for the given surgical procedure by entering a given patient's characteristics into the formula, and generating the suggested surgical port placement positions for the given patient.

The surgical port placement system 10 may be used in a number of surgical procedures. An exemplary list is shown herein.

### General Surgery: Robotic Assisted Laparoscopic/Laparoscopic

### Hiatus Hernia Repair

### Nissan Fundoplication

### Esophagectomy

### Partial Esophagectomy

### Gastrectomy

### Partial Gastrectomy

### Band Procedure

### Gastric Sleeve

### Gastric Bypass

### Cholecystectomy

### Partial Hepatectomy

### Partial Pancreatectomy

### Whipples Procedure

### Right Hemicolectomy

### Descending Hemicolectomy

### Colectomy

### Cecectomy

### Spleenectomy

### Duodenectomy

### Ventral Hernia Repair

All hernia repairs
Small bowel resection

### Urologic Surgery: Robotic Assisted Laparoscopic/Laparoscopic

### Adrenalectomy

### Partial Nephrectomy

### Radical Nephrectomy

### Pyeloplasty

### Ureteroureterostomy

### Ureteral Reimplantation

### Ureterocalylicostomy

### Nephroureterectomy

### Radical Cystectomy

### Partial Cystectomy

### Lymph Node Dissection for Bladder, Prostate, Kidney, Testicular Cancers

### Ureterolithotomy

### Pyelolithotomy

### Anatrophic Nephrolithotomy

### Neobladder Reconstruction

### Donor Nephrectomy

### Orthotopic Bladder Formation

### Non Orthotopic Bladder Formation

### Varicocelectomy

### Removal of Intraabdominal Testicle

### Radical Cystoprostatectomy

### Radical Prostatectomy

### Simple Prostatectomy

### Bladder Diverticulectomy

### Bladder Suspension

### Ureterolysis

### Renal Cyst Excision

Vesicovaginal fistula repair
Ureterovaginal fistula repair
Vesicoenteric fistula repair

### Gynecological Surgery: Robotic Assisted Laparoscopic/Laparoscopic Hysterectomy

### Radical Hysterectomy

### Myomectomy

### Oopherectomy

### Salpingo-oopherectomy

### Sacroculpopexy

### Radical Oopherectomy and Lymph Node Dissection

### Tubal Ligation

### Varicocelectomy

Tubal reanastomosis

### Endometrial Ablative Procedures

### Cardiovascular Surgery: Robotic Assisted

### Laparoscopic/Laparoscopic

### Aortic Valve Surgery

### Atrial Septal Defect Closure

### Coronary Artery Bypass Surgery

### Mitral Valve Surgery

### Tricuspid Valve Surgery

### Maze Surgery

### ENT Surgery: Robotic Assisted Laparoscopic/Laparoscopic

### Thyroidectomy

### Parathyroidectomy

### Zenkers Diverticulectomy

### Zenkers Diverticulostomy

Sleep Apnea surgery
Oral Cancer surgery
Paraganglioma surgery
Schwannoma excision
Pituitary surgery
Chordoma excision

### Mastoidectomy

Sinus surgery
Cerebellopontine angle tumor resection

An objective is to establish an optimal port placement template (robotic optimal port placement template-ROPPT) for both robotic assisted laparoscopic surgery, and laparoscopic surgery, (LOPPT) laparoscopic optimal port placement template). Robotic assisted laparoscopic surgery is becoming one of the most established surgical procedures in the United States regardless of the procedure performed today. There has been a continuum of surgical procedures established as best patient practice for decades, beginning with open surgery, which has transitioned to laparoscopic and ultimately robotic assisted laparoscopic surgery.

It is an objective to establish the best placement of robotic or laparoscopic trocars or ports prior to the initiation of the surgical procedure. In so doing, the robotic procedure/laparoscopic procedure would benefit the patient and surgeon in several ways including: 1) decreasing operating room time, thereby decreasing operating room cost both directly and indirectly, e.g., 30 minutes of anesthesia time typically costing $1100; 2) helping surgeons in training, and resident surgeons decrease the time of the learning curve for robotic/laparoscopic surgery; and 3) helping new and learning surgeons as well as experienced surgeons learning new procedures use established, proven/laparoscopic robotic port placement platforms to help optimize surgical success.

In typical robotic assisted laparoscopic surgery/laparoscopic surgery, the access to “keyhole” surgery is through small openings in the skin ranging in size from 3-12 mm. Determining the position of the robotic trocars or laparoscopic ports is based on experience gained in the operating room performing hundreds if not thousands of surgical procedures. Best robotic port placements and laparoscopic port placement are based on surgeons “experience.” This comes from techniques developed over years and based on cases previously performed. No generalized planning techniques for robotic or laparoscopic surgery regarding port placement based upon Applicants' criteria has ever been established.

From Applicants' experience as a surgeon beginning both laparoscopic and robotic assisted laparoscopic surgery, the task of determining the best port placement was tantamount to the surgical case and its ultimate success. If the robotic ports were not placed appropriately in the beginning of the case, problems may ensue. This could include poor or decreased visualization of the organs being operated on, inappropriate access to major blood vessels that need to be controlled or clamped, and crisscrossing of robotic/laparoscopic arms, thereby making it extremely difficult and potentially dangerous to even continue to operate. All of these potential problems and more will lead to increased operative time and the potential complications that may ensue, such as increased blood loss and the need for transfusions, injury to adjacent organs, and the increased rate of conversion to open surgery as well as the prolonged healing that would ensure.

Reviewing the problems of newly trained robotic/laparoscopic surgeons as well as surgeons in training or those experienced surgeons who are expanding their armamentarium of surgical procedures, Applicants have developed a Robotic/laparoscopic Optimal Port Placement Template (ROPPT) (LOPPT) (See Appendix A). In performing the ROPPT, LOPPT study, Applicants have utilized both an objective and subjective scale based on a database (stored in the memory 13 of the surgical port placement system 10, for example) of patients in the study.

### Development

The database can be utilized in many aspects of robotic assisted laparoscopic surgery and laparoscopic surgery. This would include cardiovascular, gynecologic, urologic, and general surgical procedures, for example. Utilizing a database of surgical procedures, Applicants have prospectively determined the optimal port placement for robotic and laparoscopic surgical procedures.

Based on urological procedures performed, Applicants have measured port placements from bony prominences of each patient. In doing this, Applicants have been able to exempt differences for patient's body habitus, including their height, weight and body mass index.

### Example 1

### Example Patient One Height 5′8″

Weight 193 pounds

### BMI 27.6

This patient has an incidental right lower pole kidney tumor 3.1 cm in size on the posterior margin of the kidney based on computerized tomography studies. There is a single right kidney artery and vein with no radiological evidence of metastatic disease. The surgery of Robotic Assisted Laparoscopic Partial Nephrectomy was performed on the Da Vinci robotic system, as available from Intuitive Surgical, Inc. of Sunnyvale, Calif.

Post procedure measurements were compiled while the patient remained insufflated, i.e. CO₂ is being instilled into the transperitoneal cavity. This resulted in 8 measurements.

1. Tip of Iliac crest (TIC) to Robotic Arm 1—18 cm

2. Anterior Superior Iliac Spine (ASIS) to Robotic Arm 1—21 cm

3. TIC to Robotic Arm 2—9 cm

4. ASIS to Robotic Arm 2—6.5 cm

5. Xypoid Process (XP) to Camera Arm—19 cm

6. Symphysis Pubis to Camera Arm—21 cm

7. Camera Arm to Robotic Arm 1—9 cm

8. Camera Arm to Robotic Arm 2—11 cm

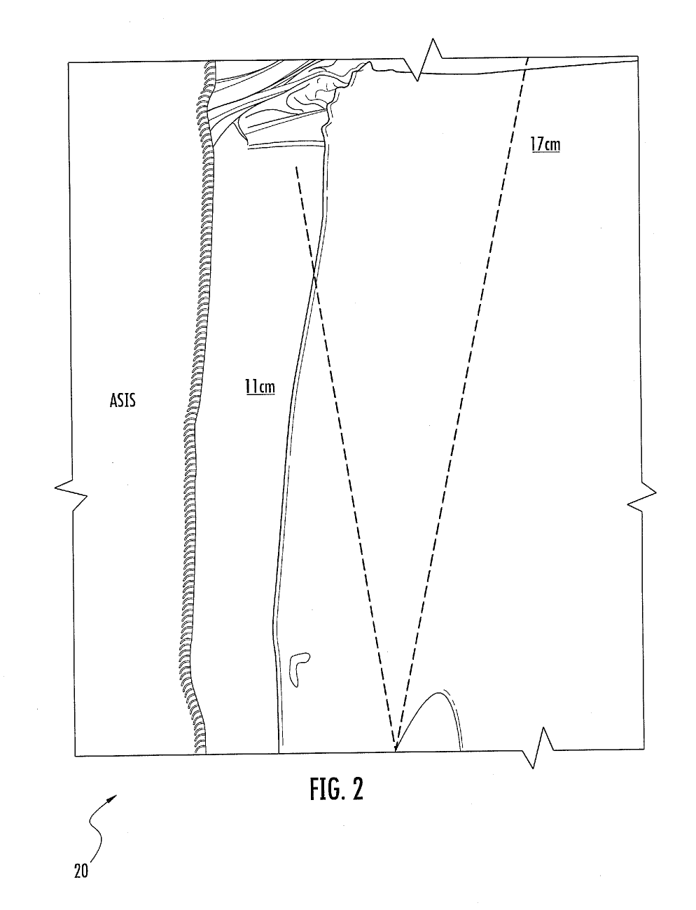

In this particular case, 3 robotic arms were used. 1) Camera Arm, 2) Robotic Arm 3) Robotic Arm 2. Referring now to FIG. 2, an image 20 of actual patient in position and the marking ports is shown.

In performing these measurements and compiling many cases over time, Applicants were able to determine that utilizing the bony prominences of patients was first, easily palpable, and secondly, reproducible in all patients independent of weight and height. This allowed an objective measurement scale that can be used from case to case. All measurements are intersected between the 2 numbers given for each patient. For example, 18 cm from the tip of the iliac crest, and 21 cm from the anterior superior iliac spine are easily measured and intersected at the distal points of measurement.

Overall, we are using a triangular pattern of measurement, which is based on a platform. In one instance, the base of the triangle is the anterior superior iliac (ASIS) to the tip of the iliac crest (TIC). In other surgical cases, the base of the triangle may be the sterna notch (SN) to the acromioclavicular joint (AC). Each base can change based upon the procedure being performed and its location.

### Subjective Scale

After each case the operating surgeon completes a subjective scoring sheet based on 7 factors. This is a negative rating scale based on the factors presented in the form supplied. Any cases that is scored at >1 out of 7 is not included in the database. This helps to eliminate any cases that can lead to unfavorable outcomes and complications. The 7 factors that are included are specific and are measured by each surgeon as inhibiting the operative procedure or not hindering the procedure.

### Exemplary Implementation

User experience in determining best sites for port placement for each patient will improve as additional participants are added to the previous array of cases, the patient data has been statistically analyzed and the formula for port placement has been refined.

Based on Applicants' database of surgical patients, the best port placements in a particular surgical case can be determined if the surgeon so desires. For example, if the doctor has a patient who is 46 years old and is suffering from an obstructed left kidney due to a ureteropelvic junction obstruction. This patient is female, weighing 205 pounds and is 5′10″ tall. Based on our database, the surgeon can determine that the camera arm after insufflation of the abdomen is best placed at:

19 cm form the XP;
22 cm from the SP;
Robotic Arm 1—10 cm from the TIC;
Robotic Arm 1—7 cm from the ASIS;
Robotic Arm 2—16 cm from the TIC; and
Robotic Arm 2—19 cm from the ASIS.

Based on the above protocol and utilizing the subjective score, the overall ROPPT gives a subjective score of 36/40. This is of course, not a perfect score and room for improvement can come from increasing the database to include female patients with a similar problem and comparable characteristics.

Alternatively, patients, for example, who are 5′8″ in height and weigh 170 pounds can be placed in Applicants' database. An optimal ROPPT measurement can be determined based on other cases that may not match directly the BMI, or height and weight of this particular patient. However, this does give the new surgeon or surgeon in training a gauge, which based on experience, may guide him/her to the appropriate and optimal port placement for this particular surgery.

As the database for optimal port placement is expanded for each particular robotic/laparoscopic surgery, the hesitation for new and inexperienced surgeons will be mitigated in part by the ability of this particular surgical aid, ROPPT, LOPPT, to allow for confidence and ease of beginning the case confidently and productively, and to complete the case accurately and successfully and as quickly as possible. This model can be utilized for robotic/laparoscopic surgery both in individual ports and in the future, single port placement involving similar robotic arms and laparoscopic arms.

### Statistical Models

For each specific surgical procedure, data will be collected on the patient position during surgery, positions of the robotic arms relative to fixed skeletal positions, basic patient data, and subjective assessments of the outcome relative to the robotic/laparoscopic performance. The patient position is determined by the procedure to be performed and the specific strategy for this robotic assisted or laparoscopic procedure. The fixed skeletal positions may comprise three or more locations related to bony prominences (unambiguous, repeatable determination of locations). The patient data includes height, weight, BMI, girth (chest, waist and hip measurements, as appropriate for the procedure), and notations of patient specific issues (e.g., prior surgeries in the same vicinity). Finally, subjective data is to be collected regarding the performance of the robotic/laparoscopic procedure during the surgery, including camera arm positioning for viewing, robotic arms for accessing the target, robotic arm port interferences, and an overall assessment of the robotic relative to absence of problems and potential for future improvement.

From the standpoint of developing statistical models, the data collection process will yield values of several independent and dependent variables that can be used to relate surgical outcomes to the situations facing the surgeon at the onset of the procedure. Potential independent variables for modeling purposes are:

X1: patient height (inches)
X2: patient weight (pounds)
X3: patient BMI (mass/height-squared; kg/sq·m. or 703 lb/sq·in.)
X4: patient chest measurement (inches)
X5: patient waist measurement (inches)
X6: patient hip measurement (inches)
X7: patient prior surgery in the vicinity (=1 for yes; =0 for no)
X8: patient age (years)
X9: patient gender (categorical)

This set of observational data is of interest in its own right as it potentially sheds indirect light on the physical characteristics related to specific surgeries. It is of direct interest as to the extent to which these patient specific characteristics are related to the surgical outcomes as assessed by the surgeon. Several models will be considered to relate the subjective response variables to these patient characteristics.

The following represent potential dependent variables:

Y1: camera positioning score
Y2: target access score
Y3: arm interference score
Y4: overall score (incorporating Y1, Y2 and Y3)
Y5: problem indicator (no problem=0; repositioning of arm(s)=1)

Standard regression models could be used for relating each of Y1 through Y4 to the Xi variables. Additionally, a Naïve Bayes Learning machine could be used to process the data. For an indicator variable, such as Y5, then a logistic regression model is more appropriate. Based on the initial wave of data collected by a surgeon who places the robotic arms using their inherent expertise, the statistical models could be used to assess the relative importance of patient characteristics on surgical outcomes (e.g., short heavy patients may be more likely to have surgical outcome issues than low body fat large individuals).

The data collection process will also involve determining physical locations of the arms relative to the target and fixed skeletal locations. Initially, the camera and robotic/laparoscopic arm positions are based on the surgeon's current expertise without resort to statistical models. For future less-experienced surgeons or those undergoing training, some statistical models could be developed to assist in the initial placement of the robotic/laparoscopic arms. Thus, in the course of the initial surgeries, specific measurements of arm placement will be made and recorded for later assessment. In particular, at least three positions on the skeleton (specified bony prominences) relevant to the procedure to be performed will be identified. These locations and an origin position (possibly the target position if it is not in the span of the plane of the skeletal points) will establish a three-dimensional coordinate system to keep track of the patient body position, the target location, and the robotic arm positions relative to the target and body location. Ultimately, the measurements taken in the data collection phase (e.g., TIS to arm1, ASIS to arm 2, etc.) will be used to obtain the configuration.

For each procedure recorded in the database, sufficient measurements must be made to recreate mathematically all of the physical positions of interest. For those surgical events with a problem-free outcome, the associated data could then be used to develop a predictive model of the initial robotic arm placements, allowing, of course, for surgeon confirmation prior to commencement of the procedure. Statistical models for placement should be updated as additional data is collected.

Aside from the statistical considerations, it is presumed that the patient is positioned so that the high-resolution camera lens has a nearly direct line of site (unobstructed view, possibly facilitated by gentle displacement of obstructing organs or tissues) to the area where the procedure(s) is/are to take place. The camera arm placement (the lens location) will be placed through a specified port location with an initial location within the insufflated zone that is close enough for visualization, but not so close as to interfere with the robotic/laparoscopic arms or preclude easy access for assistant ports. Likewise, the robotic/laparoscopic arms may require direct access to the region where the procedure is to take place. The anesthetized patient is positioned to facilitate access of the arms to the location where the procedure will take place. If only one robotic arm is to be used for the procedure (e.g. straightforward removal of a small tumor), then the camera position may be offset from a distal line of sight so that the working robotic arm has direct access.

The objective of collecting measurement data is to develop a model for predicting the placement of the camera and arms as a function of the pre-operative measurements of TIC to ASIS (both left and right sides), weight and height. A predictive formula under consideration is:

Y=ƒ(Xβ)+ε,

where

Y=(y1, y2, y3, y4, y5, y6) is a vector of measurements from

y1=ASIS to Camera

y2=TIC to Camera

y3=ASIS to Arm #1

y4=TIC to Arm #1

y5=ASIS to Arm #2

y6=TIC to Arm #2,

X is a matrix of the predictor variables including the variables

x1=TIC to ASIS (left)

x2=TIC to ASIS (right)

x3=weight

x4=height

x5=category of surgery (e.g., 1 if robotic hysterectomy; 0 if laparoscopic hysterectomy),

ƒ is a function (possibly non-linear) relating the response to the predictor variables, β is a vector of parameters to be estimated from the data, and ε an error term vector. For response variables considered individually (one specific measurement at a time), the rows of X correspond to individual patient surgeries.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the present disclosure.

APPENDIX A
Port Placement Template Data Form

| | |
|---|---|
| Patient ID: | Date of Surgery: |

| | | |
|---|---|---|
| Surgeon: | | Sex ( ) M ( ) F |
| Height ( ) ft ( ) in | Weight ( ) lbs | BMI: |
| Type of Surgery: | | |

MEASUREMENTS

| | | | |
|---|---|---|---|
| Camera To: | Anterior Superior Iliac Spine* | L ( ) cm | R ( ) cm |
| | Tip of Iliac Crest | L ( ) cm | R ( ) cm |

| | |
|---|---|
| Robotic/Lap Arm 1 | ( ) cm |
| Robotic/Lap Arm 2 | ( ) cm |
| Robotic/Lap Arm 3 | ( ) cm |

| | | | |
|---|---|---|---|
| Tip of Iliac Crest | ASIS* | L ( ) cm | R ( )cm |
| Tip of Iliac Crest | Robotic/Lap Arm 1 | L ( )cm | R ( ) cm |
| Anterior Superior Iliac Spine | Robotic/Lap Arm 1 | L ( ) cm | R ( ) cm |
| Tip of Iliac Crest | Robotic/Lap Arm 2 | L ( )cm | R ( ) cm |
| Anterior Superior Iliac Spine | Robotic/Lap Arm 2 | L ( ) cm | R ( ) cm |
| Tip of Iliac Crest | Robotic/Lap Arm 3 | L ( )cm | R ( ) cm |
| Anterior Superior Iliac Spine | Robotic/Lap Arm 3 | L ( ) cm | R ( ) cm |

SCORING (Circle Number of All That Apply and Score x/7)

| | | |
|---|---|---|
| Camera - Port Replaced, not usable | 1 | Total |
| Camera - Too Close to Target | 2 | |
| Camera - Too Far from Target | 3 | |
| Robotic/Lap Arms - Too Close to each other (clashed) | 4 | |
| Robotic/Lap Arms - Too Far Apart (Hit table, bean bag, | 5 | |
| etc.) | | |
| Robotic/Lap Arms - Unable to Reach Target | 6 | |
| Robotic/Lap Arms - 3^{rd} Arm of Little or No Use | 7 | /7 |

Comments: Please elaborate on above negative findings.

That which is claimed is:

1. A surgical port placement system to be adjacent a surgical table, the surgical port placement system comprising:
a surgical port placement device comprising a memory and processor cooperating therewith for
storing a plurality of parameter sets respectively associated with a plurality of past surgical procedures,
generating a surgical port placement model based upon the plurality of parameter sets,
receiving a given parameter set for a given surgical procedure for a given patient, the given parameter set comprising physical characteristics of the given patient, and
generating at least one surgical port placement position for the given patient for the given surgical procedure based upon the given parameter set and the surgical port placement model; and
a direction tool coupled to said surgical port placement device and for indicating the at least one surgical port placement position on the given patient on the surgical table.

2. The surgical port placement system of claim 1 wherein the physical characteristics of the given patient comprise positions for boney prominences on the given patient, a weight for the given patient, and a height for the given patient.

3. The surgical port placement system of claim 1 wherein the plurality of parameter sets respectively associated with the plurality of past surgical procedures comprises the physical characteristics of a respective patient, and a plurality of performance metrics for a respective surgical port placement position for the respective patient.

4. The surgical port placement system of claim 1 wherein the generating of the at least one surgical port placement position for the given patient for the given surgical procedure comprises generating a plurality of vectors, each vector extending from a physical point on the given patient to the at least one surgical port placement position.

5. The surgical port placement system of claim 1 wherein the generating of the surgical port placement model comprises using a Naïve Bayes Learning machine to process the plurality of parameter sets respectively from the plurality of past surgical procedures.

6. The surgical port placement system of claim 1 wherein said surgical port placement device comprises an input interface coupled to said processor for receiving the given parameter set for the given surgical procedure for the given patient.

7. The surgical port placement system of claim 1 wherein said direction tool comprises a light source for illuminating the at least one surgical port placement position on the given patient on the surgical table.

8. The surgical port placement system of claim 1 wherein said direction tool comprises a display monitor for indicating the at least one surgical port placement position on the given patient on the surgical table.

9. A surgical port placement device comprising:
a memory and processor cooperating therewith and for storing a plurality of parameter sets
respectively associated with a plurality of past surgical procedures,
generating a surgical port placement model based upon the plurality of parameter sets,
receiving a given parameter set for a given surgical procedure for a given patient, the given parameter set comprising physical characteristics of the given patient, and
generating at least one surgical port placement position for the given patient for the given surgical procedure based upon the given parameter set and the surgical port placement model.

10. The surgical port placement device of claim 9 wherein the physical characteristics of the given patient comprise positions for boney prominences on the given patient, a weight for the given patient, and a height for the given patient.

11. The surgical port placement device of claim 9 wherein the plurality of parameter sets respectively associated with the plurality of past surgical procedures comprises the physical characteristics of a respective patient, and a plurality of performance metrics for a respective surgical port placement position for the respective patient.

12. The surgical port placement device of claim 9 wherein the generating of the at least one surgical port placement position for the given patient for the given surgical procedure comprises generating a plurality of vectors, each vector extending from a physical point on the given patient to the at least one surgical port placement position.

13. The surgical port placement device of claim 9 wherein the generating of the surgical port placement model comprises using a Naïve Bayes Learning machine to process the plurality of parameter sets respectively from the plurality of past surgical procedures.

14. The surgical port placement device of claim 9 further comprising an input interface coupled to said processor for receiving the given parameter set for the given surgical procedure for the given patient.

15. A method for determining at least one surgical port placement position for a given patient for a given surgical procedure, the method comprising:
storing a plurality of parameter sets respectively associated with a plurality of past surgical procedures;
generating a surgical port placement model based upon the plurality of parameter sets;
receiving a given parameter set for the given surgical procedure for the given patient, the given parameter set comprising physical characteristics of the given patient; and
generating at least one surgical port placement position for the given patient for the given surgical procedure based upon the given parameter set and the surgical port placement model.

16. The method of claim 15 further comprising using a direction tool for indicating the at least one surgical port placement position on the given patient on the surgical table.

17. The method of claim 15 wherein the physical characteristics of the given patient comprise positions for honey prominences on the given patient, a weight for the given patient, and a height for the given patient.

18. The method of claim 15 wherein the plurality of parameter sets respectively associated with the plurality of past surgical procedures comprises the physical characteristics of a respective patient, and a plurality of performance metrics for a respective surgical port placement position for the respective patient.

19. The method of claim 15 wherein the generating of the at least one surgical port placement position for the given patient for the given surgical procedure comprises generating a plurality of vectors, each vector extending from a physical point on the given patient to the at least one surgical port placement position.

20. The method of claim 15 wherein the generating of the surgical port placement model comprises using a Naïve Bayes Learning machine to process the plurality of parameter sets respectively from the plurality of past surgical procedures.
